(19) United States
(12) Patent Application Publication
(10) Pub. No.: US 2016/0038405 A1
(43) Pub. Date: Feb. 11, 2016

(54) EFFERVESCENT MULTI-VITAMIN/MINERAL ADDITIVE FOR COFFEE AND TEA

(71) Applicant: Murcada Health LLC, Minneapolis, MN (US)

(72) Inventor: Kevin Murphy, Minneapolis, MN (US)

(21) Appl. No.: 14/822,479

(22) Filed: Aug. 10, 2015

Related U.S. Application Data
(60) Provisional application No. 62/035,280, filed on Aug. 8, 2014.

(51) Int. Cl. A61K 9/46; A23F 5/26; A23L 2/52; A23F 3/18; A23L 1/302; A23L 1/304

(52) U.S. Cl. CPC A61K 9/0007; A23L 1/302; A23L 1/304; A23L 2/52; A23F 3/18; A23F 5/267; A23V 2002/00

(57) ABSTRACT

A water soluble micronutrient composition that infuses vitamins and minerals into coffee and tea designed to maximize bio-absorption in consideration of pH levels, temperature and caffeine. The composition contains water-soluble and oil-soluble vitamins and minerals in bioavailable forms contained in a composition consisting of 1-50% of a vitamin blend with 1-35% of one or more minerals selected from the group consisting of calcium, magnesium, iron, zinc, copper and manganese, 10-55% citric acid, 5-35% of one or more alkali or alkaline earth metal bicarbonates or carbonates, 1-20% flavoring agent, 0.1-2% of a sweetening agent. When dissolved in liquid a specific amount of micronutrients are infused into the drink by way of a soluble, bioavailable form in the amount of approximately 25-100% of the US RDA of multiple vitamins and trace minerals along with a desired flavor to enhance the coffee, tea or other liquid.

### RELATED APPLICATIONS

The present application claims the benefit of previously filed U.S. Provisional Application 62/035,280, filed Aug. 8, 2014 entitled, “EFFERVESCENT MULTI-VITAMIN/MINERAL ADDITIVE FOR COFFEE AND TEA”, which is incorporated herein by reference.

### BACKGROUND

This disclosure relates to enhancing the nutritional value of coffee and tea as an additive of a water soluble composition of vitamins and minerals for infusing in coffee and tea.

Health awareness has increased the general public's knowledge and understanding of the role vitamins and minerals play in our body's overall well-being. As a result, an increasing number of people supplement their daily diet with pills containing certain amounts of combined vitamins and minerals (“multivitamins”). The vast majority of vitamin and mineral supplements for human consumption are formulated as capsules or pills. Food supplement programs have also been implemented to enrich common food to increase the likelihood of achieving recommended amounts of vitamin and mineral intake. Most people neither get the recommended daily amounts through their diet nor do they supplement their diets with said pills or capsules. According to the USDA, more than 80 percent of women and more than 70 percent of men consumed less than two-thirds of the RDA for one or more nutrients. Many people in need of supplemental vitamins and minerals have difficulty swallowing capsules or pills, or, dislike swallowing pills in general. There are also many vitamins and minerals that are needed but are not included in enriched or fortified foodstuffs. Therefore, it is impractical to consume fortified foods with the sole intent of obtaining the value of their vitamin and mineral contents.

Furthermore, multi-vitamins in pill or capsule form are not absorbed as efficiently as the same are in liquid form. Depending on the pill composition, up to 90% of the actual ingredients are passed through a human's digestive system without being absorbed. In liquid form, generally more than 80% of all micronutrients are absorbed in the body.

At the same time, it is estimated that the number of daily coffee and tea drinkers is approximately two thirds of the US population. Many of these 200 million coffee and tea drinks consumed each day are enhanced with flavor yet the drink contains nearly zero nutritional value. Although many flavor options exist and many multivitamin options exist, there is no combined delivery method of both multivitamin and flavor specifically formulated to supplement coffee and tea beverages particularly in consideration of the pH level and temperature of coffee and tea as well as the presence of caffeine.

### SUMMARY

A water soluble micronutrient composition that infuses vitamins and minerals into coffee and tea designed to maximize bio-absorption in consideration of pH levels, temperature and caffeine is herein described. The composition contains water-soluble and oil-soluble vitamins and minerals in bioavailable forms contained in a composition including (by weight) 1-50% of a vitamin blend with 1-35% of one or more minerals such as calcium, magnesium, iron, zinc, copper and manganese, 10-55% citric acid, 5-35% of one or more alkali or alkaline earth metal bicarbonates or carbonates, 1-20% flavoring agent, 0.1-2% of a sweetening agent. When dissolved in liquid a specific amount of micronutrients are infused into the drink by way of a soluble, bioavailable form in the amount of approximately 25-100% of the US RDA of multiple vitamins and trace minerals along with a desired flavor to enhance the coffee, tea or other liquid. Multiple delivery methods and flavors are available and disclose herein

As suggested in U.S. Pat. No. 4,725,427, vitamins and minerals are more bioavailable to humans if administered in a water-soluble form. One readily available delivery method of vitamins and minerals in a water-soluble form is in effervescent composition. For consumer appeal, the effervescent multivitamin should impart properties to the coffee and tea similar to those properties imparted by adding traditional flavoring including aroma, taste, appearance and mouth/feel.

Therefore, a need exists for an effervescent composition that includes a multivitamin and flavor profile for the purpose of enhancing, simultaneously, the nutritional value and flavor of coffee and tea. This effervescent composition shall be in tablet or measured dose-single serving powder form; it will dissolve in coffee and tea beverages; infuse in said beverage a defined and formulated amount of vitamins and minerals; and impart a defined and formulated flavor profile.

The concepts detailed below lies primarily in the fact that the vitamins, minerals and flavor profiles, as combined to provide effervescent composition are stable, compatible and, when dissolved in coffee and tea, are readily bioavailable. The United States Government has established recommended daily allowances for most essential vitamins and minerals (U.S. RDA) and a portion or all of these allowances may be contained in each serving portion of the granules of these concepts.

It is not, therefore, the intent to discuss or explain the need for each vitamin or mineral to be utilized. What is important is that the vitamin or mineral be made available in a form which may be readily assimilated into biological tissues. Otherwise, no matter how much of a preparation is consumed it would be of limited or no benefit.

Vitamins are usually distinguished or classified as being water-soluble or fat-soluble. The fat-soluble vitamins are the A,D,E and K vitamins and are usually measured in International Units(IU). They are generally administered in an oil base. The water-soluble vitamins are the C and various B vitamins and are measured in milligrams.

The minerals capable of forming amino acid chelates for which a U.S. RDA has been established are calcium, iron, magnesium, zinc and copper. Manganese is also considered essential although no U.S. RDA has been established. In addition, other minerals such as cobalt, vanadium, molybdenum, tin, nickel, selenium and chromium are also considered to play essential roles in life's processes. Any or all of these metals, as amino acid chelates, may be utilized in the present concepts. *(U.S. Pat No. 4,725,427 4:6-48).

### DESCRIPTION OF THE PREFERRED EMBODIMENTS

The term “effervescent composition” as used herein means any powdered or non-powdered solid and includes, without limitation, a conventional tablet, a crystal, a pellet, a granule, a grain, a cake, or a flake, regardless of size or geometric shape.

Generally, the concepts presented herein encompass an effervescent composition for direct use as an additive in hot beverages such as a cup of coffee or tea. These beverages can be served at a temperature of 160 to 185 degrees Fahrenheit. The effervescent composition can include at least one vitamin additive, at least one mineral additive, at least one pH stabilizing agent, at least one effervescence agent, and at least one solubility agent. The effervescent composition may or may not include a specific flavor profile. The effervescence agent includes sufficient effervescence to separate the tablet upon placement of the tablet in the coffee or tea. The pH stabilizing agent stabilizes the pH of the coffee or tea into which the tablet is to be placed or which forms part of the tablet. The solubility agent facilitates rapid dissolution of the tablet in the coffee or tea into which the tablet is to be placed. The vitamin additive supplements the vitamin content of the coffee or tea. The mineral additive supplements the mineral content of the coffee or tea. Various other ingredients and agents may be included in the tablet to enhance flavor, improve mouth feel, improve nutrition, enhance foam production, or achieve any other desired characteristics or results.

Example ingredients to enhance flavor may be natural or artificial and include, but are not limited to almond, amaretto, brown sugar, butter pecan, butter rum, butterscotch, caramel, chai, chocolate, cinnamon, coconut, coffee, crème de menthe, English tea, espresso, French vanilla, ginger, green tea, hazelnut, honey, Irish cream, macadamia nut, maple, mocha, peanut butter, peppermint, pumpkin, vanilla.

Example percentages of components by weight in the composition can fall in a range of: Vitamin additive 1-5%; Mineral additive 5-15%; Flavor additive 10-20%; Base Reactor(s) 15-30%; Acid Reactor(s) 10-30%; Binding Agent 10-30%; Lubricant(s) 5-10%.

The multivitamin composition formula embodies in the effervescent composition is prepared by blending the various ingredients in powdered form with mineral powdered formulation process adequately detailed in prior art. The vitamins to be blended may be in various forms. For example, one may obtain Vitamin C in the form of ascorbic acid. Similarly, one may use niacin in the form of nicotinic acid or niacinamide.

Whatever molecular form of vitamin and mineral is used it is essential that it be in powdered form. The vitamins will be selected from the group consisting of Vitamin C and any or all of the vitamins making up the B complex. B complex vitamins include thiamine (B.sub.1), riboflavin (B.sub.2), niacin (B.sub.3), pyridoxine (B.sub.6), pantothenic acid (B.sub.5), folic acid, biotin, choline, para-amino benzoic acid and inositol. Cobalamin (B.sub.12) may added with the water soluble vitamins or along with the fat-soluble ones as described below. The powdered water soluble vitamins and minerals are blended and screen sifted. The dry screened vitamins are then blended with a binding agent to the desired concentration.

In a separate container the fat-soluble vitamins are dissolved in an alcohol solvent consisting of ethanol and propylene glycol. The solvent will consist of a major portion of ethanol and a minority of propylene glycol. Into this solvent is dissolved appropriate amounts of fat-soluble vitamins as oil solutions. Vitamin A palmitate, Vitamin D.sub.2, Vitamin E from any source such as d-alpha tocopherol, d-1 alpha tocopherol acetate, and Vitamin K may be added in desired portions. The concentrations of vitamins in their oil solutions may vary as may the amounts of oil added to the alcohol solvent. Generally speaking, the solvent will comprise the major portion of the dissolved mixture and may vary from about 50 to 75% of the mixture with the oil containing the vitamins comprising from about 25 to 50%.

Specific amounts of the oil soluble vitamins dissolved in the alcohol solvent are then blended with the mixture of the dry water-soluble vitamins. Once mixed and thoroughly blended this vitamin premix is air dried thereby evaporating a major portion of the ethanol. The resulting powdered mixture is screened through a larger screen than used for the dried water-soluble vitamin mixture and stored in air and water tight containers for subsequent blending with additional ingredients.

The powdered vitamin premix and mineral mixture are combined in desired proportions and thoroughly blended to provide predetermined amounts of vitamins and minerals for each dosage unit of approximately two to six grams.

The remaining ingredients may be blended in any desired order. Therefore, the following order is exemplary only and may be rearranged to meet the operational facilities in which the formulation is prepared. To the blended vitamin and mineral mixture is added a combination of citric acid, potassium and/or sodium carbonates and bicarbonates, flavoring agents and sweeteners. The amount of flavoring agents and sweeteners used are subject to considerable variations and will not be more than necessary to provide the desired flavor and sweetness to be released from the composition when dissolved in coffee and tea. The particular flavor utilized is not critical to the proper functioning or the concepts presented herein as long as it imparts a desirable flavor to the final product.

Citric or malic acid (“Acid Reactors”) powder is present in significant concentrations. This provides sufficient acid to react with the bicarbonate salts to release carbon dioxide through effervescence. In addition, any excess citric or malic acid renders the resulting effervescent solution of vitamins and minerals acidic which enhances the flavor of the product.

The bicarbonate salts (“Base Reactors”) may be either sodium or potassium bicarbonate or a mixture thereof to comprise the base reactor. Also, other salts capable of releasing carbon dioxide in the presence of an acid may also be utilized. Typically, any suitable alkali or alkaline earth bicarbonate or carbonate may be utilized. The citric acid is preferably first blended with the vitamin and mineral mixture. The flavoring and sweetening agents may then be added followed by addition of the carbonate or bicarbonate salts. In the alternative, the bicarbonates or carbonates may be blended in with the citric acid powder followed by the addition of the flavoring and sweetening agents.

When appropriate amounts of these have all been blended, sorbitol, xyitol, dextrose or lactose, (“Binding Agents”) is added to provide the final formulation having the desired concentration of vitamins and minerals. The excipient is added such that about a two to six gram dose of the total formulation will provide up to 100% of the U.S. RDA of one or more vitamins and up to 50% of the U.S. RDA of one or more trace minerals. Generally, this requires adding between about 5 to 25% excipient based on the weight of the total formulation. This is in addition to the lactose utilized in preparing the vitamin premix as previously described. The powdered formulation thus obtained may be utilized, as is, or may be further formulated into granules or pressed into tablets. In addition, other ingredients such as lubricants, antioxidants, herbal extracts, herbal powders or other additives may be incorporated into the formulation.

The actual amount of vitamins and minerals in each dosage unit of effervescent composition may vary greatly. However, it is preferable to formulate the composition to contain between 50 to 100% of the U.S. RDA of one or more vitamins and 20-50% of the U.S. RDA of one or more trace minerals such as iron, zinc, manganese and copper. No U.S. RDA has been established for manganese. However, the Food and Nutrition Board of the National Academy of Sciences-National Research Council in 1979 proposed an RDA for manganese of 4.0 mg. For purposes of this disclosure that dosage shall be considered to be a U.S RDA for manganese. The scope of the invention however, should not be limited solely to the above ranges and is deemed to extend to any effervescent water soluble combination of vitamins and amino acid chelates formulated as described herein.

As previously stated, a dosage unit will normally consist of about two to six grams. When used as a powder, the formulation will be packed in containers of specific, single dose size. Tablets can be pressed into the desired dosage unit size. In some instances, it may be desirable to have tablets in smaller sizes such that two or more tablets comprise a unit dosage. Appropriate tableting aids such as binders, lubricants and the like may be utilized in pressing the tablets. Tablets can be packaged in unit size dosages such as foil pouches or be packaged in quantities in bottles or cans.

For use, a dosage unit of the product need merely be added to an appropriate amount of coffee or tea, i.e. 6-8 fluid ounces, and allowed to dissolve accompanied by the release of carbon dioxide through effervescence. The effervescent action uniformly disperses all ingredients in the formulation and aids in keeping all of the various vitamins and minerals in solution.

The pH of the solution after a unit dosage of composition has been dissolved therein may be regulated by the ratio of citric acid to bicarbonate contained in the composition. An excess of citric acid plus the release of carbon dioxide into the solution will obviously contribute to an acid pH. However, by having an excess of bicarbonate ions, the pH may be raised to a neutral or even basic pH if desired.

In one embodiment, the composition can include an herbal extract or other ingredient to counteract any vitamin and mineral absorption inhibitors present in caffeine. For example, the herbal extract can be avena sativa or equisetum.

Other modifications may also be made without departing from the scope of the concepts presented herein. It may be desirable to make a composition containing only vitamins or only minerals. If so, the above description would be modified by removing either the vitamin pre-blend or the minerals from the formulation and the remainder of the ingredients would be adjusted accordingly.

Various embodiments of the invention have been described above for purposes of illustrating the details thereof and to enable one of ordinary skill in the art to make and use the invention. The details and features of the disclosed embodiment[s] are not intended to be limiting, as many variations and modifications will be readily apparent to those of skill in the art. Accordingly, the scope of the present disclosure is intended to be interpreted broadly and to include all variations and modifications coming within the scope and spirit of the appended claims and their legal equivalents.

1. A water soluble micronutrient (vitamin and/or mineral) composition for direct use as an additive in coffee and tea, comprising:
at least one of a vitamin additive, a literal additive and a flavor additive;
at least one pH stabilizing agent that stabilizes a pH of the liquid into which the tablet is to be placed;
at least one effervescence agent having sufficient effervescence to separate the tablet or powder upon placement in the coffee and tea; and
at least one solubility agent that facilitates expeditious dissolution of the composition in the coffee and tea into which it is placed; wherein a percentage of components in the tablet are in a range of:
Vitamin additive 1-5%;
Mineral additive 5-15%;
Flavor additive 10-20%;
Base Reactor(s) 15-30% Acid Reactor(s) 10-30%;
Binding Agent 10-30%;
Lubricant(s) 5-10%.

2. The effervescent vitamin and mineral composition of claim 1 wherein the composition comprises a tablet and a dosage unit of said tablet is about two to six grams.

3. The effervescent vitamin and mineral composition of claim 2 wherein a dosage unit of said composition contains between about 25 and 100% of the U.S. RDA of one or more vitamins included in the composition and between about 10 and 50% of the U.S. RDA of one or more trace minerals.

4. The effervescent vitamin and mineral composition of claim 1 wherein the composition is in the form of granules.

5. The effervescent vitamin and mineral composition of claim 1 wherein the composition is in the form of a tablet.

6. The effervescent vitamin and mineral composition of claim 1 wherein the composition includes herbal extracts to counteract any vitamin and mineral absorption inhibitors present in caffeine.

7. The effervescent vitamin and mineral composition of claim 1, wherein the flavoring additive includes at least one of almond, amaretto, brown sugar, butter pecan, butter rum, butterscotch, caramel, chai, chocolate, cinnamon, coconut, coffee, crème de menthe, English tea, espresso, French vanilla, ginger, green tea, hazelnut, honey, Irish cream, macadamia nut, maple, mocha, peanut butter, peppermint, pumpkin, vanilla.

8. A method of preparing an effervescent composition of water-soluble and oil-soluble vitamins and minerals for use in coffee or tea, wherein a percentage of components in the tablet are in a range of:
Vitamin additive 1-5%;
Mineral additive 5-15%;
Flavor additive 10-20%;
Base Reactor(s) 15-30% Acid Reactor(s) 10-30%;
Binding Agent 10-30%;
Lubricant(s) 5-10%.

9. The method of preparing an effervescent composition of claim 8, wherein said composition is packaged in dosage size units containing about 2 to 6 grams.

10. The method of preparing an effervescent composition of claim 8, wherein each dosage size unit contains about 1 to 100% of the U.S. RDA of one or more of the vitamins contained therein and about 1 to 100% of the U.S. RDA of one or more trace minerals.

11. The method of preparing an effervescent composition of claim 8, wherein the effervescent powder obtained is formulated in the form of tablets by pressing a powder with a tableting aid.
